(19) United States
(12) Patent Application Publication
(10) Pub. No.: US 2021/0246109 A1
(43) Pub. Date: Aug. 12, 2021

(54) POTENT AND SELECTIVE INHIBITORS OF CYTOCHROME P450

(71) Applicant: University of Kentucky Research Foundation, Lexington, KY (US)

(72) Inventors: Edith C. Glazer, Lexington, KY (US); David K. Heidary, Lexington, KY (US)

(21) Appl. No.: 17/174,336

(22) Filed: Feb. 11, 2021

Related U.S. Application Data
(60) Provisional application No. 62/975,107, filed on Feb. 11, 2020.

(51) Int. Cl. C07D 277/24; C07D 401/04; C07D 401/14; C07D 277/28; C07D 239/26; C07D 213/30; C07C 43/285; C07C 255/16; C07D 213/56; C07D 235/18; C07D 239/74; C07D 215/14; C07F 15/00; A61P 43/00

(52) U.S. Cl. CPC C07D 277/24; C07C 2601/16; C07D 401/14; C07D 277/28; C07D 239/26; C07D 213/30; C07C 43/285; C07C 255/16; C07D 213/56; C07D 235/18; C07D 239/74; C07D 215/14; C07F 15/0053; A61P 43/00; C07D 401/04

(57) ABSTRACT

Inhibitors of the enzyme cytochrome P450 (CYP), including 1B1 (CYP1B1), 1A1 (CYP1A1) and 19A1 (CYP19A1) are provided, and are useful in medical applications. Disclosed are highly potent and selective compounds that can be used in chemoprevention to ameliorate malignant changes induced by CYP, or to aid in treatment, including restoration of chemotherapeutic efficacy.

### RELATED APPLICATIONS

This application claims priority from U.S. Provisional Application Ser. No. 62/975,107 filed Feb. 11, 2020, the entire disclosure of which is incorporated herein by this reference.

### GOVERNMENT INTEREST

This invention was made with government support under grant number 5R01GM107586 awarded by the National Institutes of Health. The government has certain rights in the invention.

### TECHNICAL FIELD

The present invention relates to relates to small molecule inhibitors of the enzyme cytochrome P450 (CYP), including 1B1 (CYP1B1), 1A1 (CYP1A1) and 19A1 (CYP19A1) for use in medical applications. In particular, the invention provides highly potent and selective compounds that may be used in chemoprevention to ameliorate malignant changes induced by CYP, or to aid in treatment, including restoration of chemotherapeutic efficacy.

### INTRODUCTION

Cytochrome P450 (CYP)-1B1 (CYP1B1), -1A1 (CYP1A1), and -19A1(CYP19A1) are three of 57 unique Cytochrome P450s encoded in the human genome. CYP1B1 is not significantly expressed in the liver and other healthy tissues, but is overexpressed in tumors, and is thus considered a “universal tumor maker”.¹ More than 80 CYP1B1 single nucleotide polymorphisms (SNPs) have been identified in humans,²⁻⁴ and many induce point mutations. Pathological mutations have been found in 3-4.4% of the US population.⁵

There is strong evidence from clinical and epidemiological studies that CYP1B1 is involved in cancer initiation, progression, and chemotherapeutic resistance, and moreover, that specific mutations further enhance the malignant phenotype. CYP1B1 overexpression and point mutations result in resistance to many commonly used chemotherapeutics, such as cisplatin,⁶ daunorubicin,⁷ and taxanes.⁸⁻¹¹ Mutations may alter N-terminal proteolytic processing, which has been demonstrated to result in relocalization of the protein from the ER to the mitochondria.¹² CYP1B1 is associated with mitochondrial dysfunction, oxidative stress,¹³ and drug toxicity. This includes doxorubicin associated cardiotoxicity, which is reversed upon 1B1 inhibition.¹⁴

The association and mechanistic rationale for the role of CYP1B1 in malignant initiation is clearest for hormone related cancers, particularly for breast, ovarian, endometrial, and prostate cancer.¹⁵ CYP1B1 generally hydroxylates 17 β-estradiol at the 4 position, rather than the 2 position; this turns estrogen into a DNA damaging mutagen.^{16, 17} The metabolite profile of CYP1B1 is dependent on protein sequence; for example, the single point mutation V395L in human CYP inverts the regioselectivity from 4-hydroxylation to 2-hydroxylation of estradiol.¹⁸ Thus, SNPs in CYP are associated with alterations in metabolism. CYP metabolizes many compounds in addition to estradiol; there is the possibility of additional unknown substrates.

CYP1B1 has also been closely associated with degenerative optic neuropathy, which is one cause of blindness. CYP1B1 mutations are connected to primary congenital glaucoma (PCG) and congenital glaucoma with anterior segment dysgenesis (CG with ASD). There has also been an association of CYP variants with adult-onset primary open angle glaucoma (POAG).¹⁹ Moreover, CYP abnormalities result in irregularities in tissue collagen, including its distribution, and increased levels of oxidative stress. Decreased levels of periostin (Postn), an extracellular matrix protein that is expressed and secreted in collagen-rich tissues, is also found in patients with mutations in CYP1B1.²⁰ This has implications for both ocular development and maintenance, as well as epithelial tissue proliferation and migration with an impact on metastasis. There is also data that demonstrates that modulation of CYP1B1 can decrease hypertension, atherosclerosis, and impact adipogenesis.²¹ Selective inhibitors of CYP1B1 are thus of interest for a variety of medical applications.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compounds and methods for using such compounds. In some embodiments, the compound of the presently-disclosed subject matter is a compound of the formula:

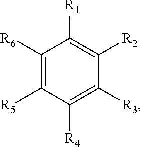

wherein R1, R4, R5, and R6 are independently selected from the group consisting of H, OH, alkoxy, thiazole, phenylthiazole, halo, alkylhalo, alkyl, alkenyl, alkynyl, alkenylpyrimidinyl, alkenylpyrazinyl, alkenylpyridinyl, cyano, alkyoxycyano, amide, benzimidazole, and alkylketone; and R2 and R3 are independently selected from the group consisting of H, OH, alkoxy, thiazole, phenylthiazole, halo, alkylhalo, alkyl, alkenyl, alkynyl, alkenylpyrimidinyl, alkenylpyrazinyl, alkenylpyridinyl, cyano, alkyoxycyano, amide, benzimidazole, and alkylketone; or R2 and R3 taken together with the atoms to which they are bound for a 6 member substituted or unsubstituted heterocycle; wherein at least one of R1-R6 is selected from the group consisting of substituted or unsubstituted thiazole, quinazolinone, (E)-4-(prop-1-en-1-yl)pyrimidine, (E)-2-(prop-1-en-1-yl)pyrazine, (E)-3-(prop-1-en-1-yl)pyridine, (E)-5-(prop-1-en-1-yl)pyrimidine, (E)-prop-1-en-1-ylbenzene, 1H-benzo[d]imidazole, quinazoline-4(3H)-one, 2-(methyleneamino)benzamide, and 4-alkoxy-3,4-dihydroquinazoline. In some embodiments, the presently-disclosed subject matter includes a method for inhibiting CYP by administering a compound as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 includes nine (9) exemplary scaffolds for the construction of CYP inhibitors.

FIGS. 2A-E includes data show small molecules selectively targeting CYP1B1. FIG. 2A includes a dose response of 547 in EROD assay in CYP1B1 cell line (blue) vs. 1A1 cell line (black). FIG. 2B includes results of a wash out experiment with 547 demonstrates that CYP activity is suppressed for >10 h. 510 is a control compound that lacks a coordinating group. FIG. 2C shows that binding of 547 (0.1 μM) causes degradation of CYP1B1. FIG. 2D shows that there is no loss in activity of CYP1A1 with 1 μM 547 was observed over 24 h. FIG. 2E shows the effect of cisplatin (CP; 100 μM) on MCF7 tumor spheroids. CP-1B1 cells do not express CYP1B1. All other conditions are following CYP1B1 induction with benzo[a]pyrene. Spheroid shown in inset.

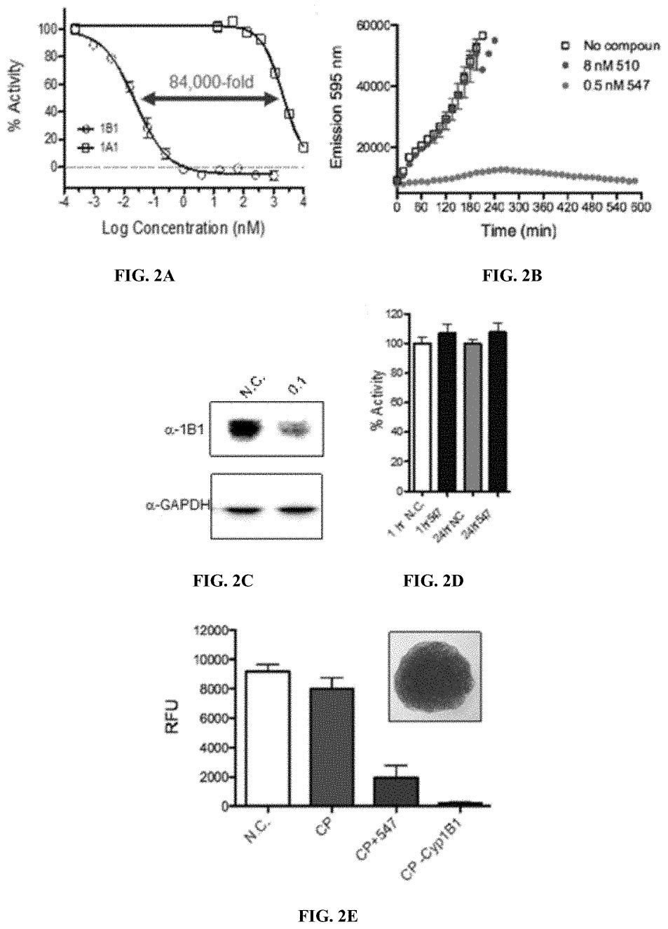

FIG. 3 shows some general synthetic schemes for construction of multiple CYP inhibitors listed herein.

FIG. 4 shows synthetic schemes for modification of scaffolds provided herein.

FIG. 5 shows synthetic routes for construction of various chalcone compounds disclosed herein.

FIG. 6 shows synthetic routes for various benzimidazole compounds disclosed herein.

FIGS. 7A and 7B show light active systems for CYP1B1 inhibition. FIG. 7A includes results associated with ruthenium (II) complex coordinated to a pyrimidine modified stilbene inhibitor 4. Different light energies are represented by their respective colors. The activity of the free ligand is shown in red. FIG. 7B includes results associated with another ruthenium (II) complex coordinated to a pyridine modified stilbene inhibitor 6. This system was developed for red light activation.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds that are potent and selective inhibitors of cytochrome P450 (CYP), and methods of using the compounds for inhibiting CYP and/or delivering with another compound or moiety having therapeutic efficacy.

The presently-disclosed subject matter includes compounds and methods for using such compounds. In some embodiments, the compound of the presently-disclosed subject matter is a compound of the formula:

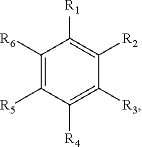

wherein R1, R4, R5, and R6 are independently selected from the group consisting of H, OH, alkoxy, thiazole, phenylthiazole, halo, alkylhalo, alkyl, alkenyl, alkynyl, alkenylpyrimidinyl, alkenylpyrazinyl, alkenylpyridinyl, cyano, alkyoxycyano, amide, benzimidazole, and alkylketone; and R2 and R3 are independently selected from the group consisting of H, OH, alkoxy, thiazole, phenylthiazole, halo, alkylhalo, alkyl, alkenyl, alkynyl, alkenylpyrimidinyl, alkenylpyrazinyl, alkenylpyridinyl, cyano, alkyoxycyano, amide, benzimidazole, and alkylketone; or R2 and R3 taken together with the atoms to which they are bound for a 6 member substituted or unsubstituted heterocycle; wherein at least one of R1-R6 is selected from the group consisting of substituted or unsubstituted thiazole, quinazolinone, (E)-4-(prop-1-en-1-yl)pyrimidine, (E)-2-(prop-1-en-1-yl)pyrazine, (E)-3-(prop-1-en-1-yl)pyridine, (E)-5-(prop-1-en-1-yl)pyrimidine, (E)-prop-1-en-1-ylbenzene, 1H-benzo[d]imidazole, quinazoline-4(3H)-one, 2-(methyleneamino)benzamide, and 4-alkoxy-3,4-dihydroquinazoline.

In some embodiments, the compound is selected from:

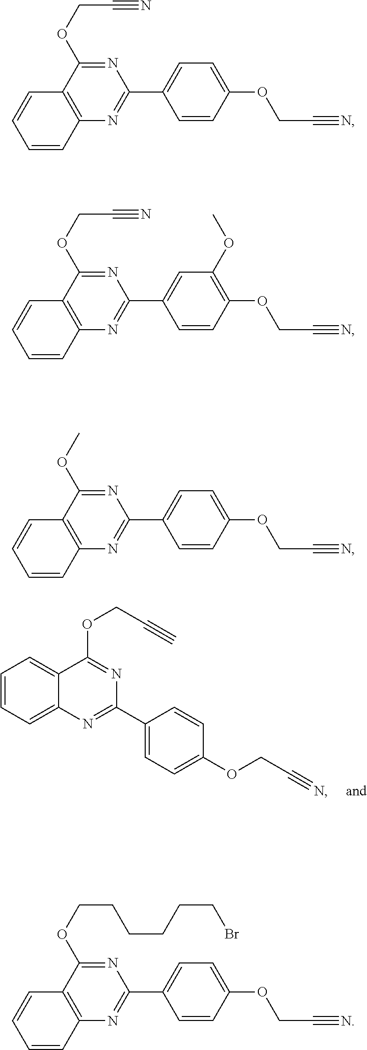 [SMILES: N#CCOc1ccc(-c2nc(OCC#N)c3ccccc3n2)cc1 ; COc1cc(-c2nc(OCC#N)c3ccccc3n2)ccc1OCC#N ; COc1nc(-c2ccc(OCC#N)cc2)nc2ccccc12 ; C#CCOc1nc(-c2ccc(OCC#N)cc2)nc2ccccc12 ; N#CCOc1ccc(-c2nc(OCCCCCCBr)c3ccccc3n2)cc1]

In some embodiments, the compound is selected from:

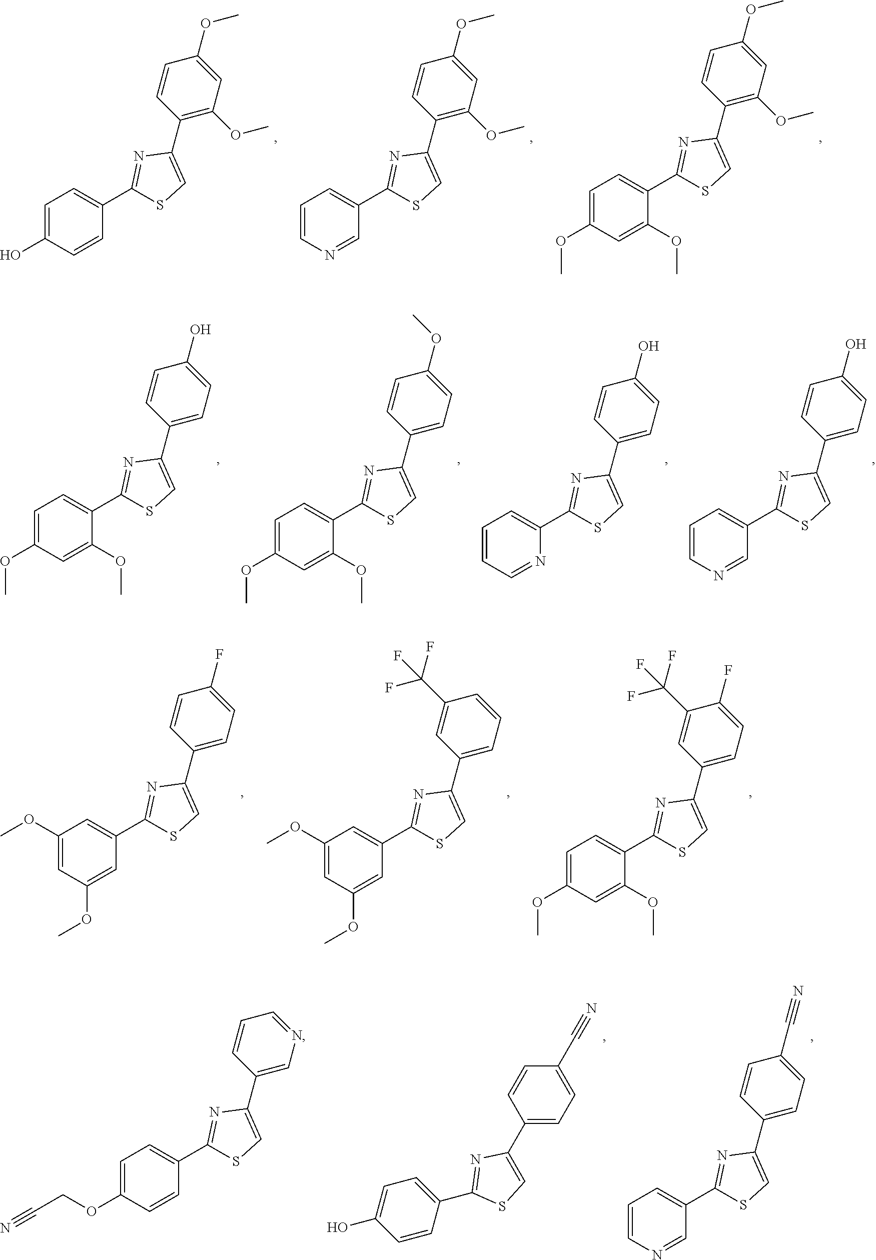

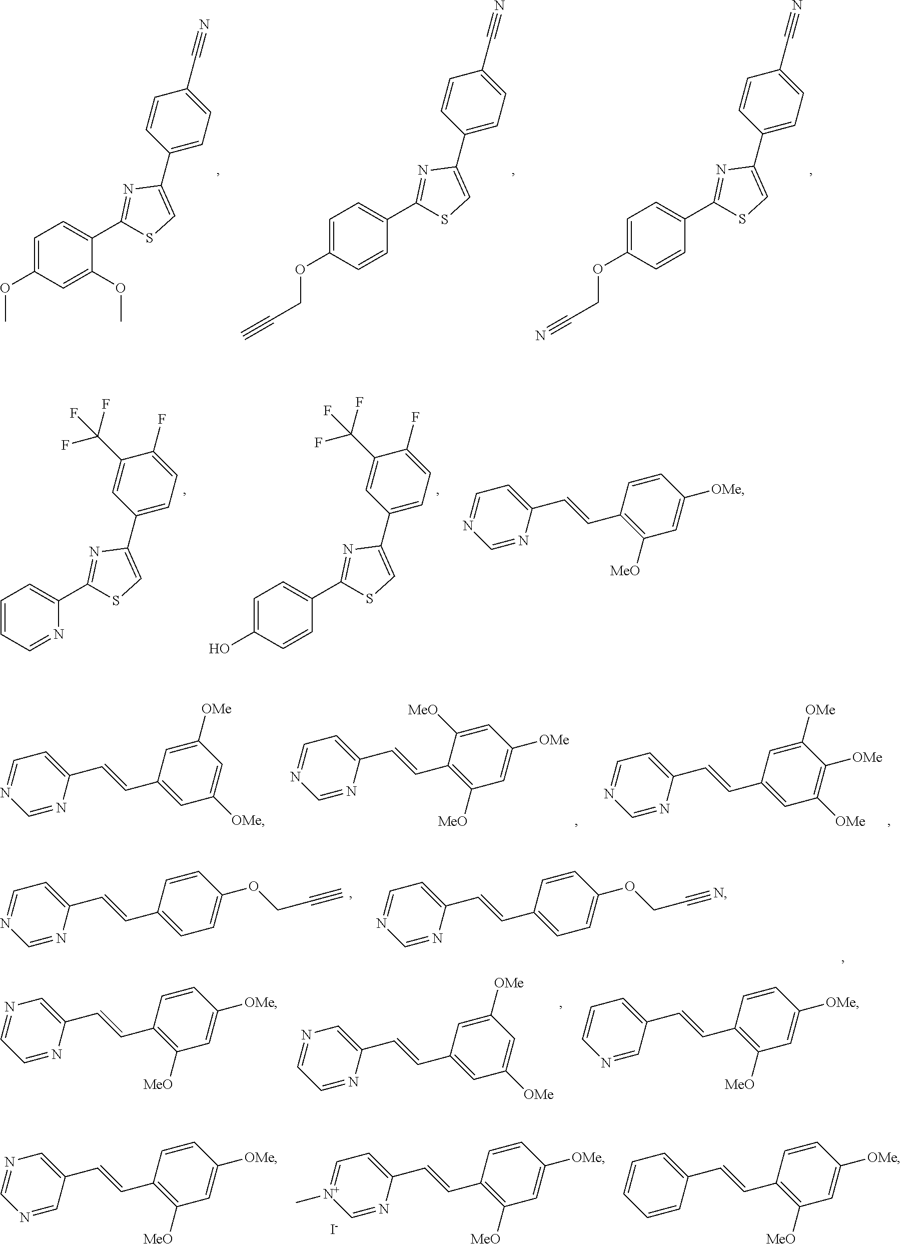

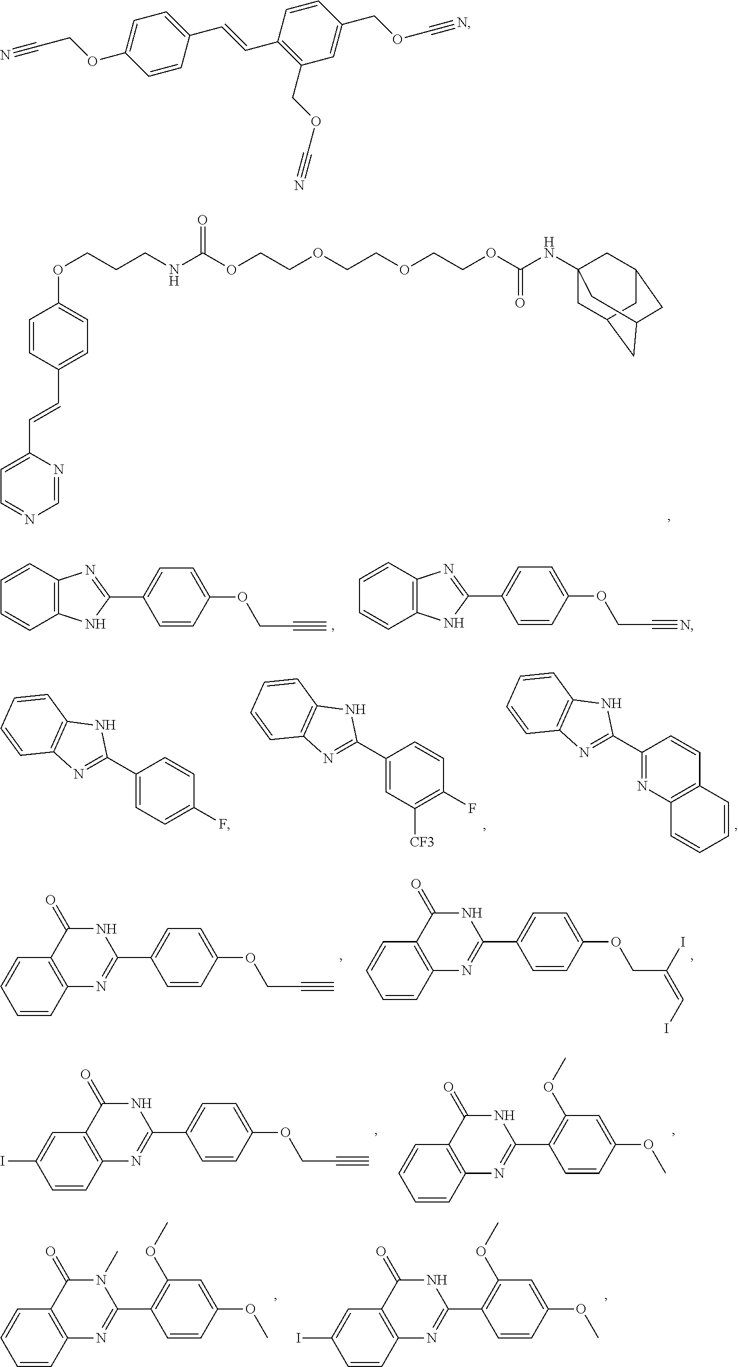

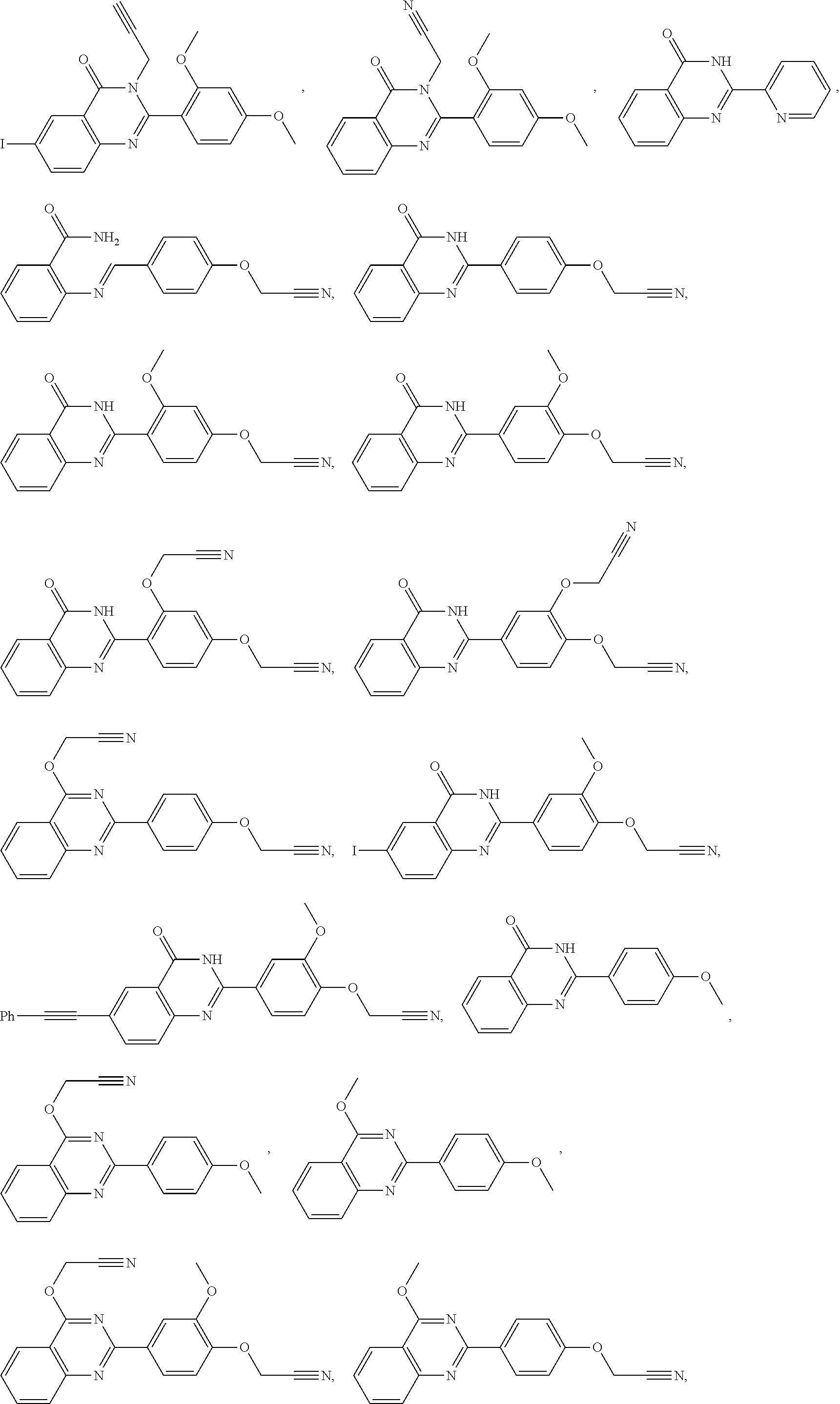

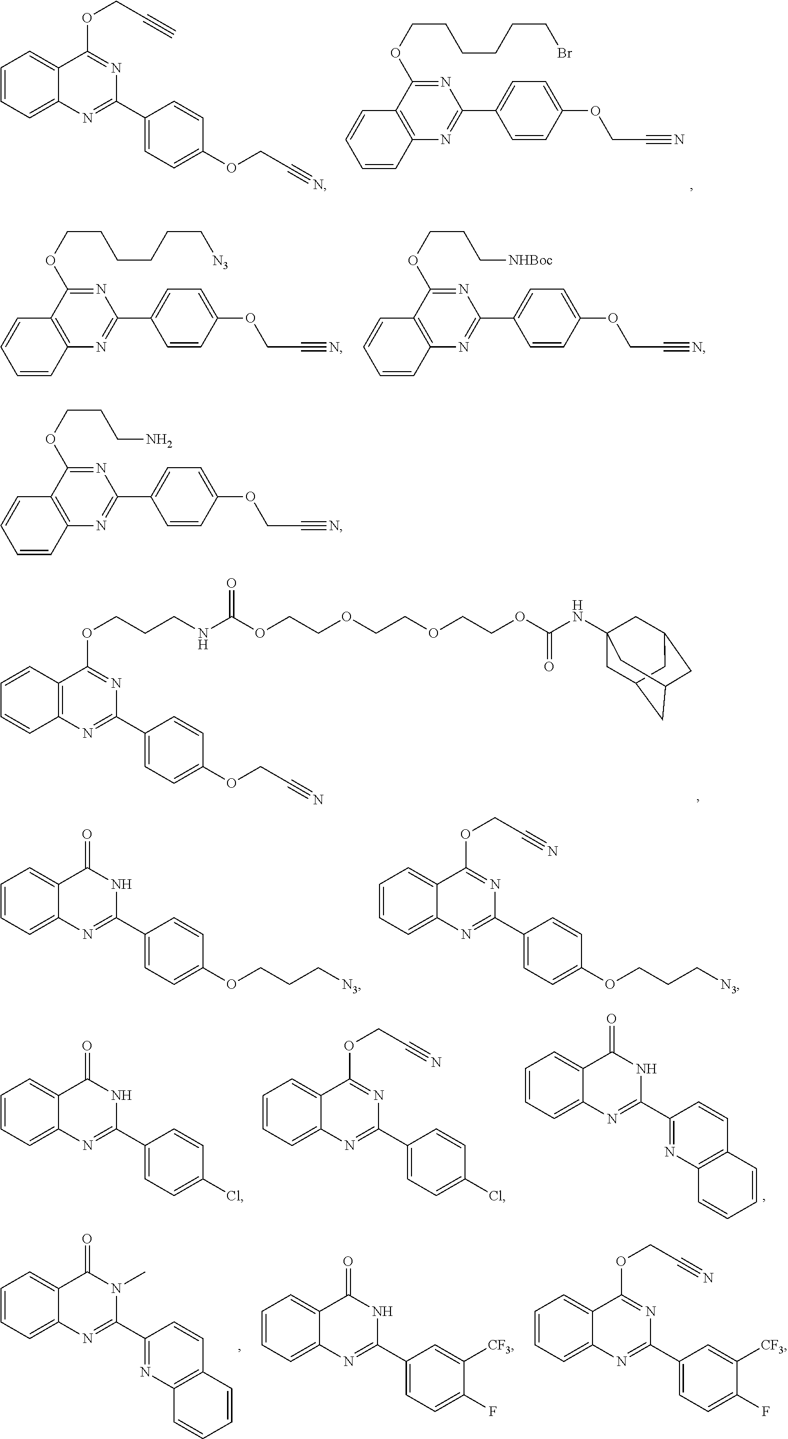

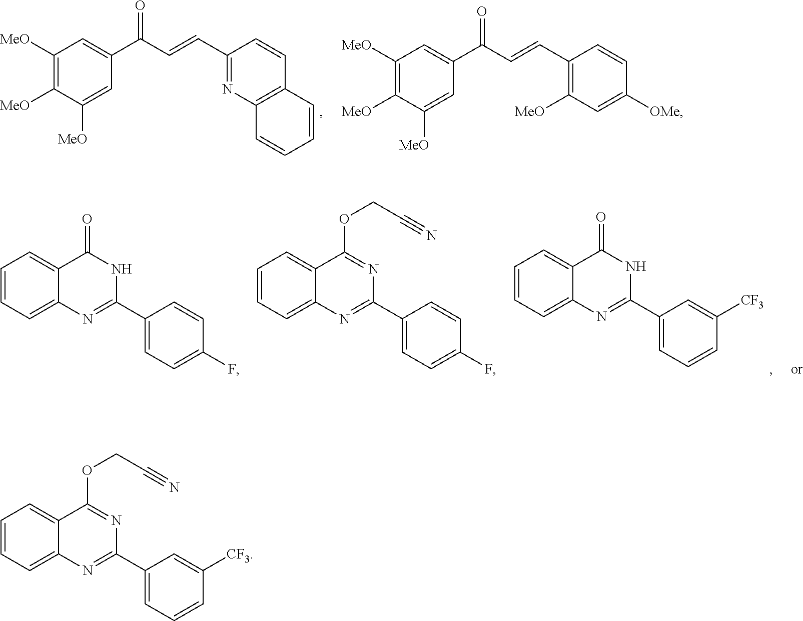 [SMILES: COc1cc(C(=O)/C=C/c2ccc3ccccc3n2)cc(OC)c1OC ; COc1ccc(/C=C/C(=O)c2cc(OC)c(OC)c(OC)c2)c(OC)c1 ; O=c1[nH]c(-c2ccc(F)cc2)nc2ccccc12 ; N#CCOc1nc(-c2ccc(F)cc2)nc2ccccc12 ; O=c1[nH]c(-c2cccc(C(F)(F)F)c2)nc2ccccc12 ; N#CCOc1nc(-c2cccc(C(F)(F)F)c2)nc2ccccc12]

In some embodiments, the compound is selected from:

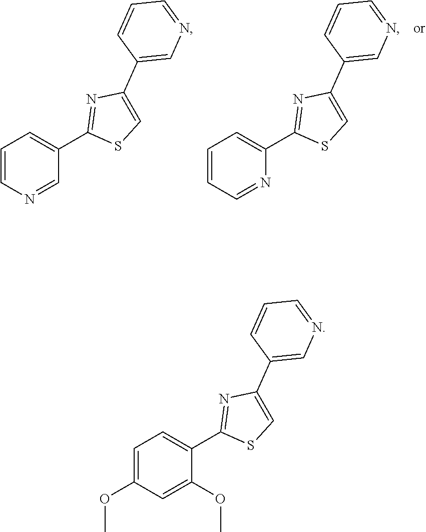 [SMILES: c1cncc(-c2csc(-c3cccnc3)n2)c1 ; c1ccc(-c2nc(-c3cccnc3)cs2)nc1 ; COc1ccc(-c2nc(-c3cccnc3)cs2)c(OC)c1]

In some embodiments, the compound is selected from:

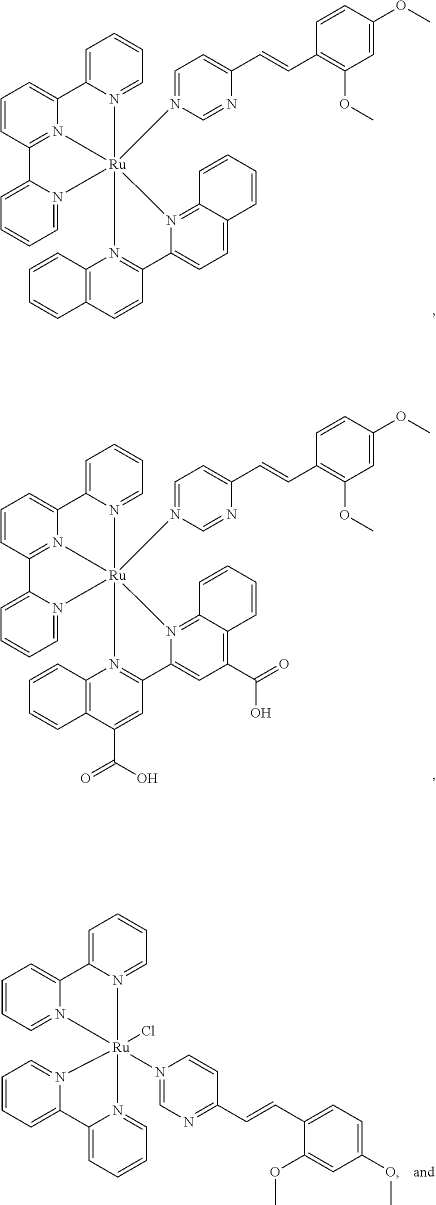

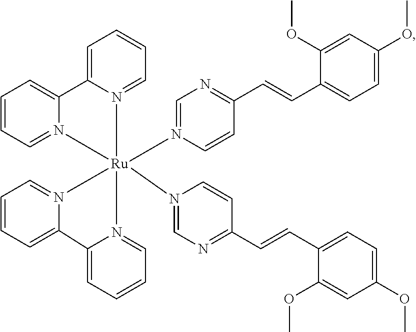

In some embodiments, the presently-disclosed subject matter includes a method for inhibiting CYP1B1 by administering to cells a compound disclosed herein.

In some embodiments, the presently-disclosed subject matter includes a method for inhibiting CYP1B1 in a subject comprising administering to the subject a therapeutically effective amount of a compound disclosed herein.

In some embodiments, the presently-disclosed subject matter includes a method for restoring the chemotherapeutic efficacy of a chemotherapeutic agent comprising administering to a patient a compound disclosed herein and a chemotherapeutic agent.

The compounds disclosed herein can include all salt forms, for example, salts of both basic groups, inter alia, amines, as well as salts of acidic groups, inter alia, carboxylic acids. The following are non-limiting examples of anions that can form salts with protonated basic groups: chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form salts of acidic groups: ammonium, sodium, lithium, potassium, calcium, magnesium, bismuth, lysine, and the like.

The analogs (compounds) of the present disclosure are arranged into several categories to assist the formulator in applying a rational synthetic strategy for the preparation of analogs which are not expressly exampled herein. The arrangement into categories does not imply increased or decreased efficacy for any of the compositions of matter described herein.

The presently-disclosed subject matter also includes pharmaceutical compositions. In some embodiments, the presently-disclosed subject matter includes pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In some embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In some embodiments, an amount suitable for inhibiting CYP1B1 will be about 1 nM to about 100 μM. It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The disclosed pharmaceutical compositions can further comprise other therapeutically active compounds.

It is understood that the disclosed compositions can be prepared from the disclosed compounds. It is also understood that the disclosed compositions can be employed in the disclosed methods of using.

Further disclosed herein are pharmaceutical compositions comprising one or more of the disclosed compounds and a pharmaceutically acceptable carrier.

Accordingly, the pharmaceutical compositions of the present invention include those that contain one or more other active ingredients, in addition to a compound of the present invention.

The above combinations include combinations of a disclosed compound not only with one other active compound, but also with two or more other active compounds. Likewise, disclosed compounds may be used in combination with other drugs that are used in the prevention, treatment, control, amelioration, or reduction of risk of the diseases or conditions for which disclosed compounds are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

In such combinations the compound of the present invention and other active agents may be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent may be coadministered, either in concomitant therapy or in a fixed combination.

In some embodiments of the presently-disclosed subject matter the compound can be employed in combination with chemotherapeutic agents.

In some embodiments, the presently-disclosed subject matter relates to a pharmaceutical composition comprising a compound disclosed herein or a pharmaceutically acceptable salt thereof or a pharmaceutically acceptable derivative thereof; and a pharmaceutically acceptable carrier.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. Such a diagnosis can be in reference to a disorder, such as cancer, and the like, as discussed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula (CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by a formula —C(O)H. Throughout this specification “C(O)” is a short hand notation for a carbonyl group, i.e., C═O.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by a formula —C(O)OH.

The term “ester” as used herein is represented by a formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by a formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by a formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is, represented by a formula -(A¹O-A²O)ₐ— where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by a formula —OH.

The term “ketone” as used herein is represented by a formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by a formula —N₃.

The term “nitro” as used herein is represented by a formula —NO₂.

The term “nitrile” as used herein is represented by a formula —CN.

The term “silyl” as used herein is represented by a formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by a formulas —S(O)A¹, S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by a formula S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by a formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by a formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by a formula —SH.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compounds disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Several methods for preparing the compounds of this invention are illustrated in the following Examples. Starting materials and the requisite intermediates are in some cases commercially available, or can be prepared according to literature procedures or as illustrated herein.

The invention identifies specific structures and functional groups that significantly increase the potency and selectivity of small molecules for inhibition of CYP1B1 activity. The mechanism underlying this inhibition may be due to direct engagement of the enzyme, or through suppression of protein production or activation of protein degradation.

Multiple scaffolds have been investigated and the selectivity and potency radically improved by the incorporation of specific functional groups and substituents. Key substituents are trifluoromethyl, fluoro, and nitrile groups.

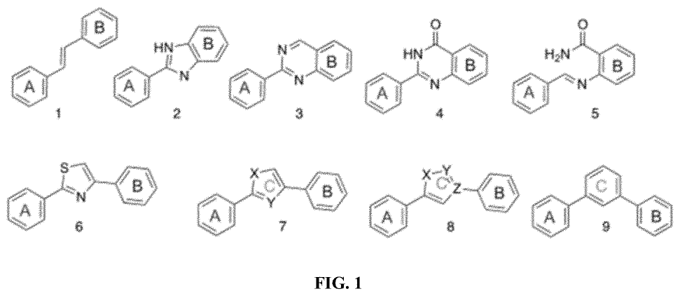

Rings A and B may contain identical substituents in the same pattern, or the same substituents in a different pattern (e.g., substituents at the 2, 4 positions on ring A and 3, 5 positions in ring B in FIG. 1). They may contain different substituents, and different substituents in different numbers on each ring and in different positions.

Rings A and B may be heterocycles, containing nitrogen(s) in any positions. These include but are not limited to pyridine, pyrazine, pyrimidine, pyridazine. Ring C may be any 5- or 6-membered ring, including but not limited to thiazole, oxazole, indole, thiophene, furan, imidazole, triazole, pyridine, pyrazine, pyrimidine, pyridazine.

Substituents at each occurrence are each independently selected from the group consisting of hydrogen, deuterium, optionally substituted alkyl, optionally substituted branched alkyl, optionally substituted cycloalkyl, optionally substituted haloalkyl, optionally substituted alkoxy, CO2R, CONR2, NR2, sulfate, sulfonate, optionally substituted aryl, optionally substituted aryloxy, optionally substituted heteroaryl, and optionally substituted heterocycle.

In addition, pendant groups may be attached to the inhibitor scaffold. These can include any and all fluorophores. They may also include systems known to induce protein degradation, such as adamantane. They may also include systems known to function as E3 ligase ligands for the construction of small molecule proteolysis-targeting chimaeras (PROTACs). These include but are not limited to pomalidomide and the VHL-ligand. Other protein degrading technologies, such as LYTAC (lysosome targeting chimera), AUTAC (autophagy-targeting chimera), and ATTEC (autophagosome-tethering compound) may be applied. Also, cell and tissue-directing groups can be appended.

### Example 2

The activity of the inhibitors was assessed in a novel cell based assay generated for the purpose of this project. Enzymatic turnover of CYP1B1 was measured using the ethoxy-resorufin-O-deethylase (EROD) assay with the fluorescent substrate, resorufin ethyl ether. Cell lines were generated where the gene for CYP1B1 alone or CYP1B1 and cytochrome P450 reductase (CPR) were both under the control of an inducible promoter. A cell line was generated for counter screening where the gene for CYP1A1 was used. CYP1A1 is the closest family member to CYP1B1.

Human liver microsomes were used as a counterscreen to experimentally determine the magnitude of inhibitor selectivity. Liver P450 proteins include CYP3A4, CYP2D6, CYP2A1 and CYP2C9, which metabolize approximately 95% of drugs in clinical use.²² These xenobiotic metabolizing CYPs are essential for regular liver function, and thus CYP1B1 inhibitors should not affect their activity. The use of pooled human liver microsomes (pHLMs) ensures that data is not biased due to variation as a result of different enzyme variants. These could be a result of gender, ethnicity, and mutation.

### Example 3

Cytotoxicity was assessed at 72 hours after compound addition using resazurin. The compounds that function as selective 1B1 inhibitors had no effect on cell health. This is an essential feature for any chemopreventative agent or molecules intended to block the detrimental action of an enzyme.

Restoration of chemotherapeutic efficacy has been reported for several CYP1B1 inhibitors⁶ and following CYP1B1 knockdown by RNAi.²³ In order to determine if CYP1B1 inhibition by the inhibitors of the current invention would affect chemotherapy resistance, CYP1B1 expression was induced in MCF7 breast cancer cells with benzo[a]pyrene, and the cells were grown into 3D tumor spheroids. Cisplatin (100 μM) did not significantly reduced cell viability in spheroids expressing CYP1B1, while they caused complete cell death in cells that don't express CYP1B1. Compound 547 (0.5 μM) was able to increase cell death by 75%. Thus, it appears that 547 restores cisplatin efficacy in CYP1B1 expressing spheroids.

TABLE 1
Selectivity profile for CYP1B1 inhibitors.

| | | | | Cyto- |
|---|---|---|---|---|
| | CYP1B1 | CYP1A1 | hLM | toxicity |
| Compound | IC₅₀ (nM) | IC₅₀ (nM) | IC₅₀ (nM) | IC₅₀ (nM) |
| ANF | 140 +/− 5 | 86 +/− 4 | >5,000 | >10,000 |
| TMS | 8.3 +/− 1 | 1500 +/− 20 | 1870 +/− 20 | 10,000 |
| GL-433 | 76 +/− 5 | 10,000 | 1100 +/− 202 | >10,000 |
| GL-543 | 4.9 +/− 0.7 | >10,000 | >10,000 | >10,000 |
| GL-547 | 0.024 +/− 0.003 | 1400 +/− 30 | 4000 +/− 0.1 | >10,000 |
| GL-581 | 0.12 +/− 0.07 | 3600 +/− 20 | — | >10,000 |

### Example 4

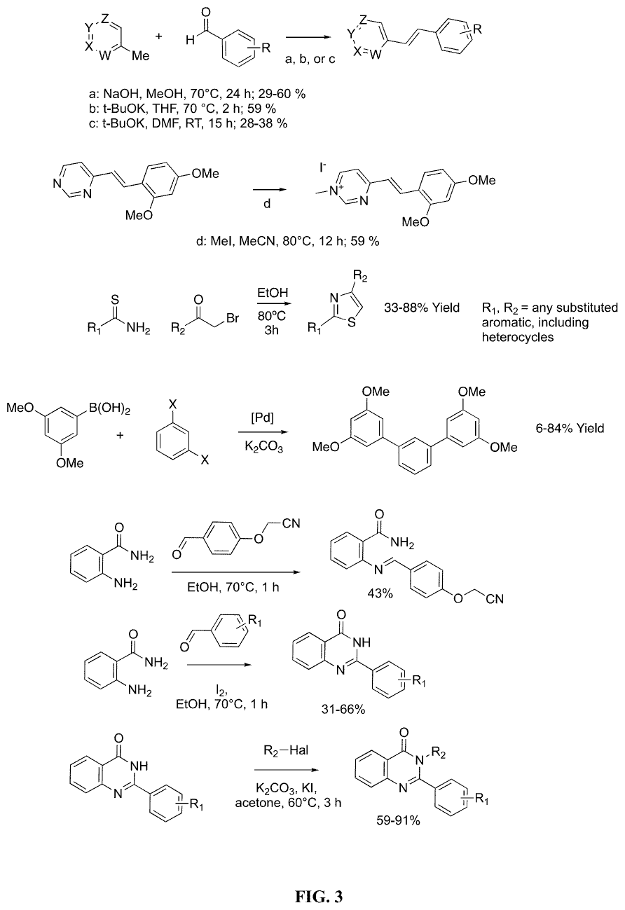

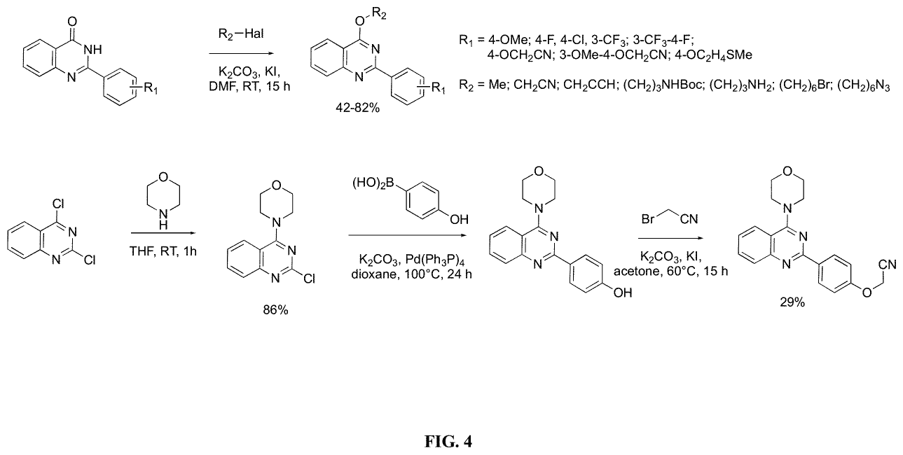

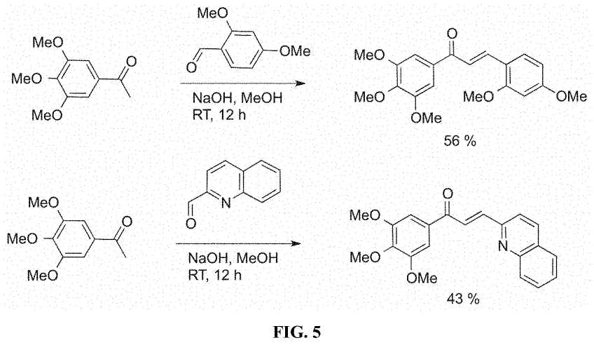

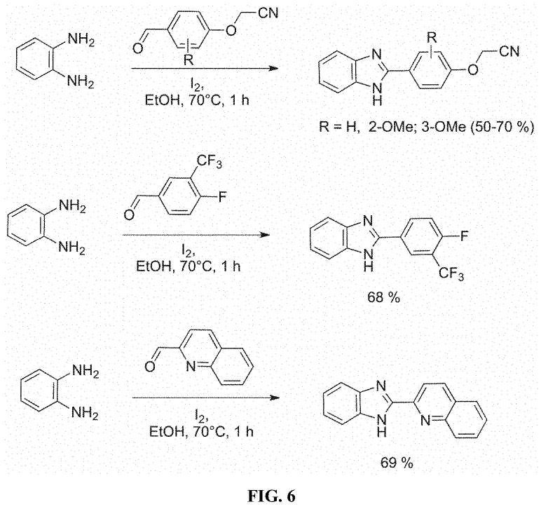

FIG. 3 includes some general synthetic schemes for construction of multiple CYP1B1 inhibitors listed herein. FIG. 4 shows synthetic schemes for modification of scaffolds provided herein. FIG. 5 shows synthetic routes for construction of various chalcone compounds disclosed herein. FIG. 6 shows synthetic routes for various benzimidazole compounds disclosed herein. One of ordinary skill in the art can readily discern the synthetic routes of all the compounds disclosed herein based on existing knowledge in the field and the schemes provided in FIGS. 3-6.

### Example 5

Structures and activities of various exemplary CYP inhibitors are set forth in Tables 2 and 3.

TABLE 2
Structures and Activities of Thiazole CYP1B1 Inhibitors

| GL | | IC₅₀ | IC₅₀ | IC₅₀ | SI 1B1: | SI 1B1: |
|---|---|---|---|---|---|---|
| Code | Structure | CYP1B1 | CYP1A1 | pHLM | 1A1 | HLM |
| 650 | 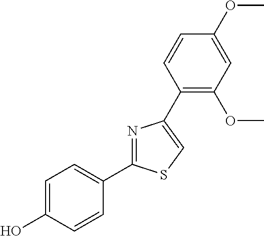 [SMILES: COc1ccc(-c2csc(-c3ccc(O)cc3)n2)c(OC)c1] | 0.0619 | 1.961 | 5.55 | 32 | 89.7 |
| 654 | 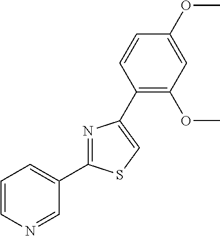 [SMILES: COc1ccc(-c2csc(-c3cccnc3)n2)c(OC)c1] | 0.0198 | 0.1512 | 0.0001 | 7.6 | 0.00082 |
| 660 | 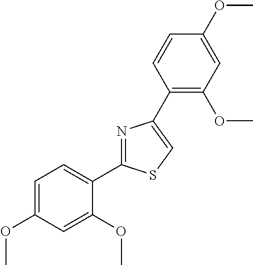 [SMILES: COc1ccc(-c2csc(-c3ccc(OC)cc3OC)n2)c(OC)c1] | 0.0062 | 5.65 | >10 | 913 | >1616 |
| 673 | 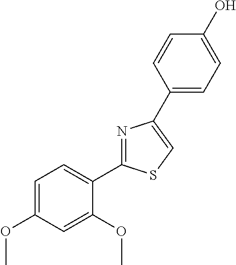 [SMILES: COc1ccc(-c2nc(-c3ccc(O)cc3)cs2)c(OC)c1] | 0.1006 | 1.984 | >10 | 19.7 | >99 |
| 679 | 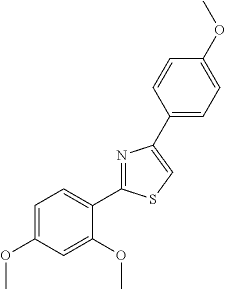 [SMILES: COc1ccc(-c2csc(-c3ccc(OC)cc3OC)n2)cc1] | 0.025 | 2.881 | >10 | 115 | >399 |
| 672 | 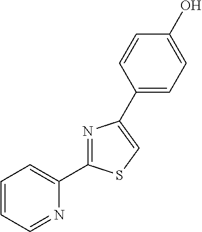 [SMILES: Oc1ccc(-c2csc(-c3ccccn3)n2)cc1] | 1.015 | 1.322 | 1.65 | 1.3 | 1.63 |
| 671 | 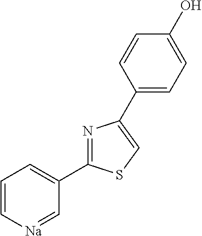 | 2.229 | 1.409 | 0.526 | 0.63 | 0.024 |
| 665 | 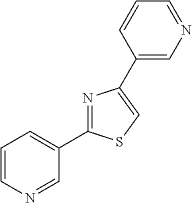 [SMILES: c1cncc(-c2csc(-c3cccnc3)n2)c1] | >10 | 0.3502 | >1 | <0.035 | <0.00025 |
| 681 | 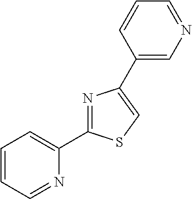 [SMILES: c1ccc(-c2nc(-c3cccnc3)cs2)nc1] | 2.393 | 0.085 | 3.243 | 0.035 | 0.14 |
| 661 | 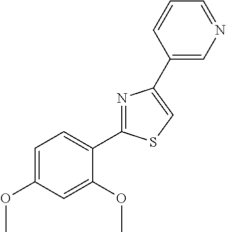 [SMILES: COc1ccc(-c2nc(-c3cccnc3)cs2)c(OC)c1] | 0.0055 | 0.0255 | −0.5 | 4.7 | 0.0015 |
| 683 | 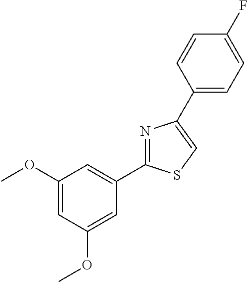 [SMILES: COc1cc(OC)cc(-c2nc(-c3ccc(F)cc3)cs2)c1] | <0.00000001 | 2.99 | >10 | >299000 | >1000000 |
| 684 | 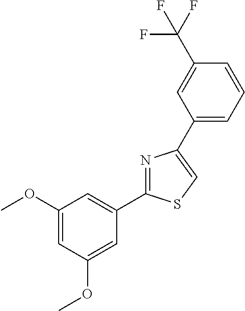 [SMILES: COc1cc(OC)cc(-c2nc(-c3cccc(C(F)(F)F)c3)cs2)c1] | <0.00000001 | 2.78 | >10 | >27800 | >1000000 |
| 674 | 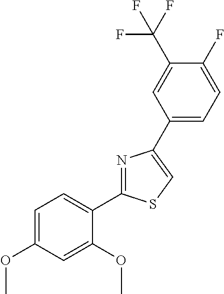 [SMILES: COc1ccc(-c2nc(-c3ccc(F)c(C(F)(F)F)c3)cs2)c(OC)c1] | 0.00098 | 5.083 | >10 | 5187 | >10204 |
| 641 | 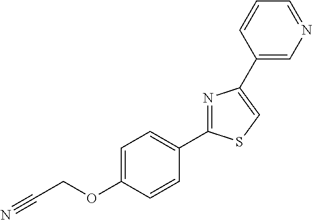 [SMILES: N#CCOc1ccc(-c2nc(-c3cccnc3)cs2)cc1] | 0.0003814 | 2.606 | NT | 6833 | |
| 649 | 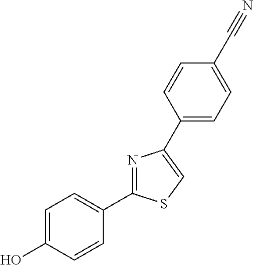 [SMILES: N#Cc1ccc(-c2csc(-c3ccc(O)cc3)n2)cc1] | 0.9307 | 16.41 | NT | 17.6 | |
| 656 | 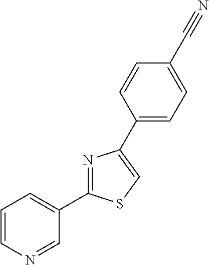 [SMILES: N#Cc1ccc(-c2csc(-c3cccnc3)n2)cc1] | 0.3906 | NT | NT | | |
| 662 | 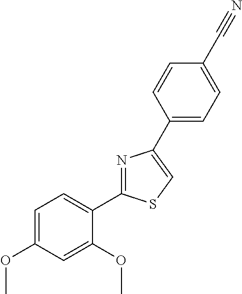 [SMILES: COc1ccc(-c2nc(-c3ccc(C#N)cc3)cs2)c(OC)c1] | 0.0000208 | 0.5254 | NT | 25259 | |
| 664 | 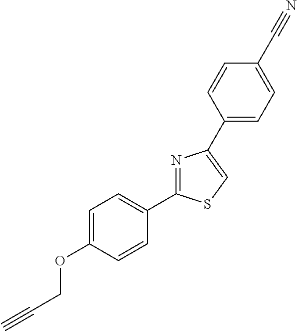 [SMILES: C#CCOc1ccc(-c2nc(-c3ccc(C#N)cc3)cs2)cc1] | 0.2022 | 0.6964 | NT | 3.44 | |
| 665 | 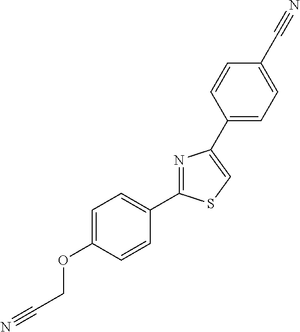 [SMILES: N#CCOc1ccc(-c2nc(-c3ccc(C#N)cc3)cs2)cc1] | <0.0001 | 1.08 | NT | >10800 | |
| 666 | 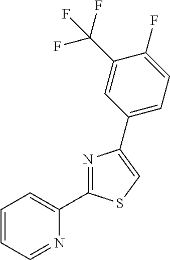 [SMILES: Fc1ccc(-c2csc(-c3ccccn3)n2)cc1C(F)(F)F] | 0.0838 | 0.2396 | NT | 2.86 | |
| 678 | 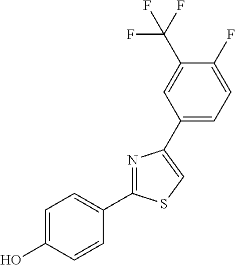 [SMILES: Oc1ccc(-c2nc(-c3ccc(F)c(C(F)(F)F)c3)cs2)cc1] | 0.2084 | 14.7 | NT | 70.5 | |

SI = selectivity Index;
pHLM = pooled human liver microsomes. All activities evaluated by EROD assay.

TABLE 3
Structure and Activities of CYP1B1 Inhibitors

| | | | | | HEK |
|---|---|---|---|---|---|
| | | | | | cyto- |
| | | | | | toxicity |
| | | | IC50 | IC50 | (μM) or |
| | CMPD | IC50 1B1 | pHLM | 1A1 | % at |
| | ID | (μm) | (μM) | (μM) | 10 μM |
| | TMS | 0.083 | 18.7 ± 0.2 | 1.5 | >10 |
| | ANF | 0.14 | >500 | 0.86 | >10 |
| 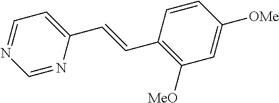 [SMILES: COc1ccc(/C=C/c2ccncn2)c(OC)c1] | GL433 | 0.076 | 0.91 ± 0.37 | 10 | >10 |
| 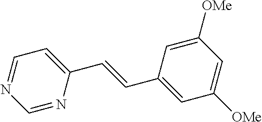 [SMILES: COc1cc(/C=C/c2ccncn2)cc(OC)c1] | GL443 | 0.76 | 2.30 ± 0.98 | >10 | 105.2% |
| 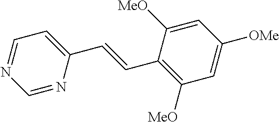 [SMILES: COc1cc(OC)c(/C=C/c2ccncn2)c(OC)c1] | GL456 | 0.21 | 0.15 ± 0.08 | 10 >10 | 93.6% |
| 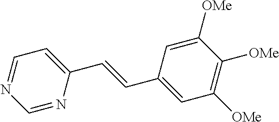 [SMILES: COc1cc(/C=C/c2ccncn2)cc(OC)c1OC] | GL465 | 0.26 | 4.40 ± 1.71 | 0.73 0.74 | 83.4% |
| 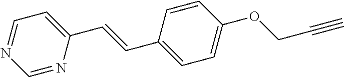 [SMILES: C#CCOc1ccc(/C=C/c2ccncn2)cc1] | GL508 | 0.1053 | 32.88 | 10 5.6 | 93.4% |
| 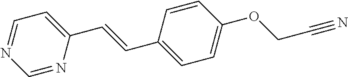 [SMILES: N#CCOc1ccc(/C=C/c2ccncn2)cc1] | GL543 | 0.0049 0.0064 | | >10 >10 | |
| 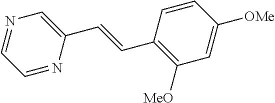 [SMILES: COc1ccc(/C=C/c2cnccn2)c(OC)c1] | GL435 | 0.51 | 1.57 ± 0.29 | 2.6 | 71.4% |
| 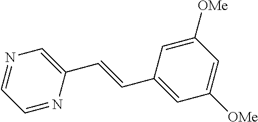 [SMILES: COc1cc(/C=C/c2cnccn2)cc(OC)c1] | GL437 | 0.66 | 5.27 ± 1.60 | 4.28 3.29 | 103.1% |
| 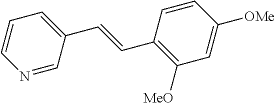 [SMILES: COc1ccc(/C=C/c2cccnc2)c(OC)c1] | GL457 | 0.31 | 0.42 ± 0.24 | 0.403 0.36 | 91.5% |
| 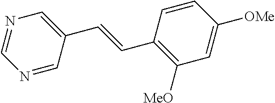 [SMILES: COc1ccc(/C=C/c2cncnc2)c(OC)c1] | GL458 | 2.51 | 2.26 ± 1.05 | 4.13 2.5 | 95.8% |
| 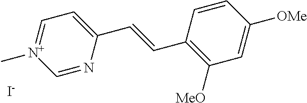 [SMILES: COc1ccc(/C=C/c2cc[n+](C)cn2)c(OC)c1 ; [I-]] | GL461 | 13.9 | — | >10 | 103% |
| 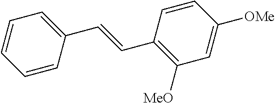 [SMILES: COc1ccc(/C=C/c2ccccc2)c(OC)c1] | GL469 | 1 | 6 | 4.46 >10 | 124.2% |
| 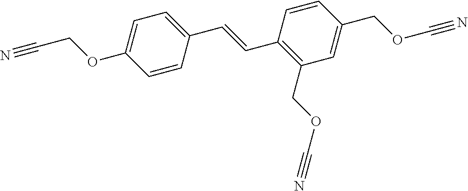 [SMILES: N#CCOc1ccc(/C=C/c2ccc(COC#N)cc2COC#N)cc1] | GL652 | 0.019 | | 1.86 | |
| 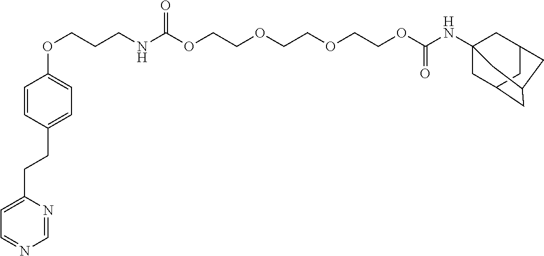 [SMILES: O=C(NCCCOc1ccc(CCc2ccncn2)cc1)OCCOCCOCCOC(=O)NC12CC3CC(CC(C3)C1)C2] | GL594 | 9.4 | | 1.1 | |
| 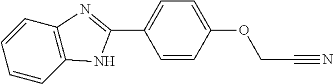 [SMILES: N#CCOc1ccc(-c2nc3ccccc3[nH]2)cc1] | GL509 | 0.316 | 8.36 | 5.5 3.5 | 75% |
| 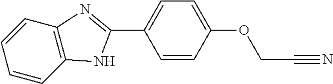 [SMILES: N#CCOc1ccc(-c2nc3ccccc3[nH]2)cc1] | GL523 | 0.03345 | | >10 >10 | 93.4% |
| 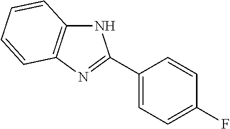 [SMILES: Fc1ccc(-c2nc3ccccc3[nH]2)cc1] | GL-719 | 3 | | 5.2 | |
| 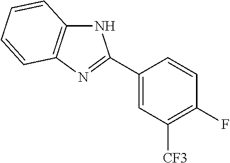 [SMILES: Fc1ccc(-c2nc3ccccc3[nH]2)cc1C(F)(F)F] | GL-706 | 0.085 | | 0.32 | |
| 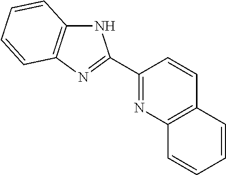 [SMILES: c1ccc2nc(-c3nc4ccccc4[nH]3)ccc2c1] | GL-707 | 0.044 | | 0.76 | |
| 581 | GL499 | 0.006 | 53.1 | | 36.8% |
| 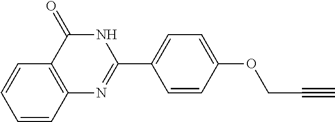 [SMILES: C#CCOc1ccc(-c2nc3ccccc3c(=O)[nH]2)cc1] | GL510 | 0.0148 0.0089 0.0109 | 0.8569 | 1.32 | 0.406 |
| 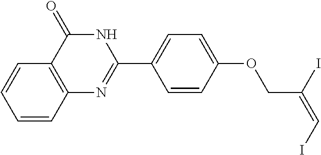 [SMILES: O=c1[nH]c(-c2ccc(OC/C(I)=C\I)cc2)nc2ccccc12] | GL511 | 0.0118 | 7.706 | >10 >10 | 64.3% |
| 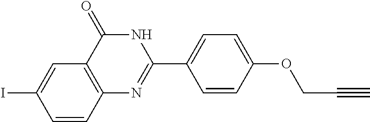 [SMILES: C#CCOc1ccc(-c2nc3ccc(I)cc3c(=O)[nH]2)cc1] | GL516 | 0.014 | 126.0 | 4.58 2.46 1.9 | 40.8% |
| 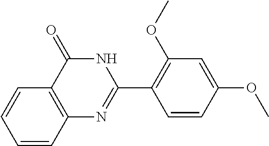 [SMILES: COc1ccc(-c2nc3ccccc3c(=O)[nH]2)c(OC)c1] | GL512 | 0.0116 | 16.9 | 2.4 1.04 | 85.3% |
| 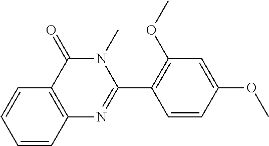 [SMILES: COc1ccc(-c2nc3ccccc3c(=O)n2C)c(OC)c1] | GL517 | >0.5 | 146.2 | >10 | 107% |
| 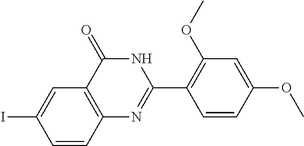 [SMILES: COc1ccc(-c2nc3ccc(I)cc3c(=O)[nH]2)c(OC)c1] | GL513 | 0.036 | 26.21 | 0.121 0.075 0.091 | 83.4% |
| 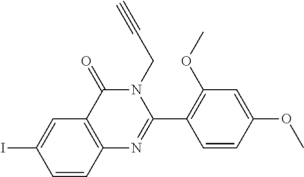 [SMILES: C#CCn1c(-c2ccc(OC)cc2OC)nc2ccc(I)cc2c1=O] | GL514 | 0.66 | 105.2 | 4.42 3.5 | 72.5% |
| 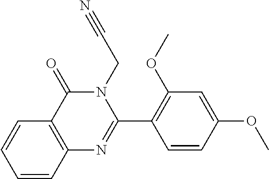 [SMILES: COc1ccc(-c2nc3ccccc3c(=O)n2CC#N)c(OC)c1] | GL519 | 0.97 | | >10 >10 | 88.7% |
| 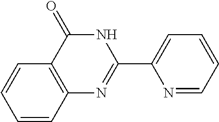 [SMILES: O=c1[nH]c(-c2ccccn2)nc2ccccc12] | GL518 | 2.44 | | | |
| 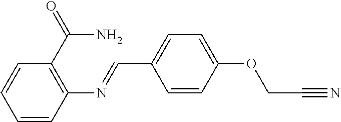 [SMILES: N#CCOc1ccc(/C=N/c2ccccc2C(N)=O)cc1] | GL542 | 0.051 | | >10 >10 | |
| 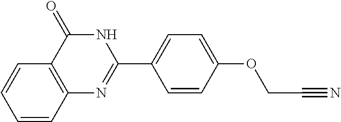 [SMILES: N#CCOc1ccc(-c2nc3ccccc3c(=O)[nH]2)cc1] | GL520 | 0.00088 0.00092 | 1.74 | 2.57 | 89% |
| 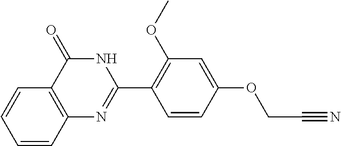 [SMILES: COc1cc(OCC#N)ccc1-c1nc2ccccc2c(=O)[nH]1] | GL521 | 0.00051 0.0013 | >10 | 1.23 1.18 | |
| 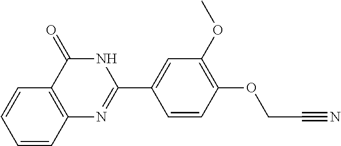 [SMILES: COc1cc(-c2nc3ccccc3c(=O)[nH]2)ccc1OCC#N] | GL522 | 0.00066 0.0025 | >10 | 2.27 0.97 | 80.6% |
| 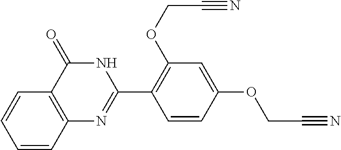 [SMILES: N#CCOc1ccc(-c2nc3ccccc3c(=O)[nH]2)c(OCC#N)c1] | GL545 | 0.00067 0.0036 | | 4.4 | |
| 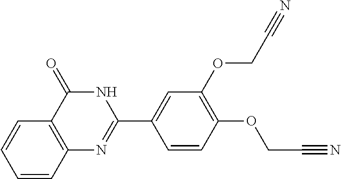 [SMILES: N#CCOc1ccc(-c2nc3ccccc3c(=O)[nH]2)cc1OCC#N] | GL546 | 0.0204 | | 3.5 | |
| 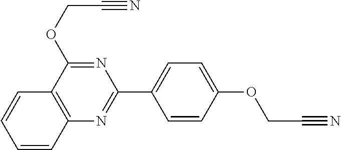 [SMILES: N#CCOc1ccc(-c2nc(OCC#N)c3ccccc3n2)cc1] | GL547 | <0.00025 | 3 | 3.73 2.69 | |
| 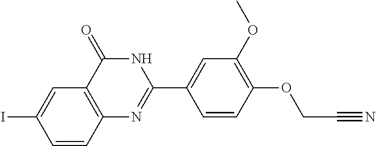 [SMILES: COc1cc(-c2nc3ccc(I)cc3c(=O)[nH]2)ccc1OCC#N] | GL551 | <0.00025 | >3 | 0.56 | |
| 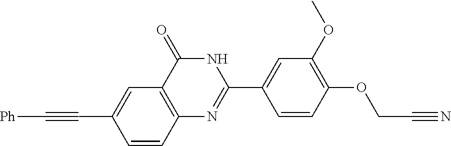 [SMILES: COc1cc(-c2nc3ccc(C#Cc4ccccc4)cc3c(=O)[nH]2)ccc1OCC#N] | GL552 | 0.0018 | | >10 | |
| 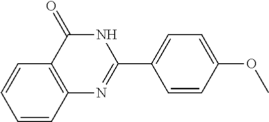 [SMILES: COc1ccc(-c2nc3ccccc3c(=O)[nH]2)cc1] | GL553 | 0.0038 | | 4.76 | |
| 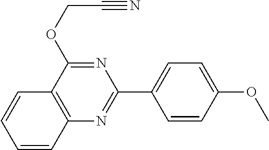 [SMILES: COc1ccc(-c2nc(OCC#N)c3ccccc3n2)cc1] | GL554 | 0.0035 | | 3.33 | |
| 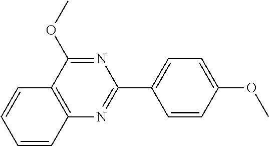 [SMILES: COc1ccc(-c2nc(OC)c3ccccc3n2)cc1] | GL556 | 0.0067 | | 2.59 | |
| 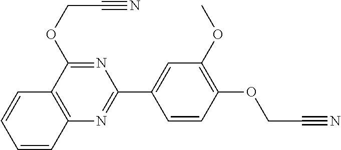 [SMILES: COc1cc(-c2nc(OCC#N)c3ccccc3n2)ccc1OCC#N] | GL557 | <0.0007 | | 1.72 | |
| 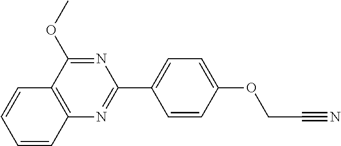 [SMILES: COc1nc(-c2ccc(OCC#N)cc2)nc2ccccc12] | GL558 | <0.0007 | | 2.46 | |
| 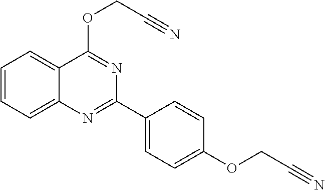 [SMILES: N#CCOc1ccc(-c2nc(OCC#N)c3ccccc3n2)cc1] | GL- 561 | <10⁻⁸ | | 3.6 | |
| 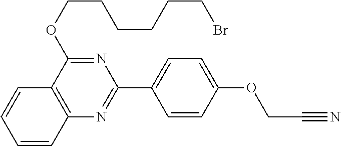 [SMILES: N#CCOc1ccc(-c2nc(OCCCCCCBr)c3ccccc3n2)cc1] | GL564 | <0.0025 | >10 | 1.94 | |
| 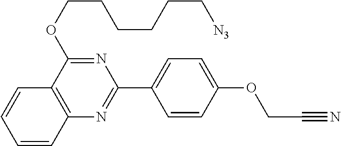 [SMILES: N#CCOc1ccc(-c2nc(OCCCCCCN=[N+]=[N-])c3ccccc3n2)cc1] | GL565 | <0.0025 | >10 | 1.87 | |
| 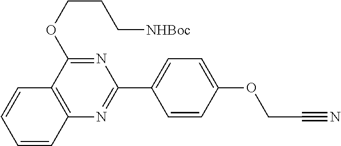 [SMILES: CC(C)(C)OC(=O)NCCCOc1nc(-c2ccc(OCC#N)cc2)nc2ccccc12] | GL571 | <0.0025 | >10 | 1.28 | |
| 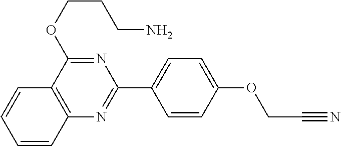 [SMILES: N#CCOc1ccc(-c2nc(OCCCN)c3ccccc3n2)cc1] | GL572 | <0.0025 | >10 | 2.12 | |
| 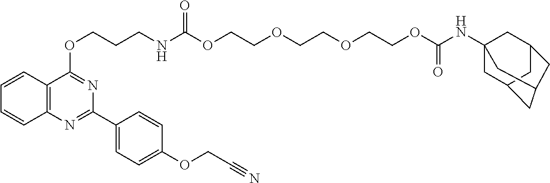 [SMILES: N#CCOc1ccc(-c2nc(OCCCNC(=O)OCCOCCOCCOC(=O)NC34CC5CC(CC(C5)C3)C4)c3ccccc3n2)cc1] | GL581 | <0.0025 | >10 | 3.58 | |
| 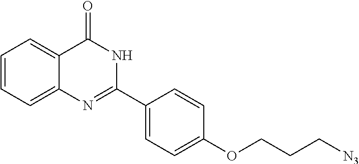 [SMILES: [N-]=[N+]=NCCCOc1ccc(-c2nc3ccccc3c(=O)[nH]2)cc1] | GL627 | 0.0061 | | | |
| 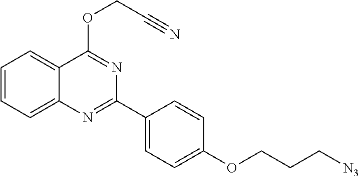 [SMILES: N#CCOc1nc(-c2ccc(OCCCN=[N+]=[N-])cc2)nc2ccccc12] | GL628 | 0.126 | | | |
| 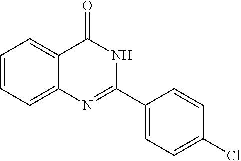 [SMILES: O=c1[nH]c(-c2ccc(Cl)cc2)nc2ccccc12] | GL642 | 0.022 | | 0.085 | |
| 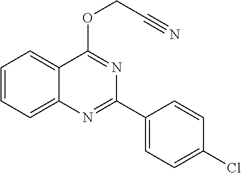 [SMILES: N#CCOc1nc(-c2ccc(Cl)cc2)nc2ccccc12] | GL643 | 0.0001 | | 0.125 | |
| 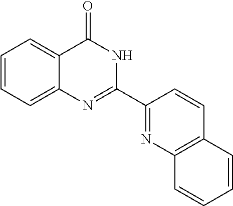 [SMILES: O=c1[nH]c(-c2ccc3ccccc3n2)nc2ccccc12] | GL651 | 0.0028 | | 1.1 | |
| 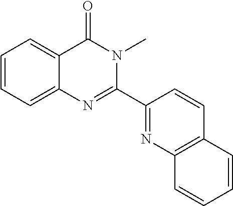 [SMILES: Cn1c(-c2ccc3ccccc3n2)nc2ccccc2c1=O] | GL659 | >1 | | >10 | |
| 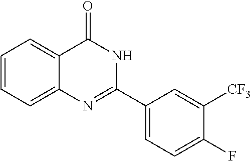 [SMILES: O=c1[nH]c(-c2ccc(F)c(C(F)(F)F)c2)nc2ccccc12] | GL-700 | 0.041 | | 2.3 | |
| 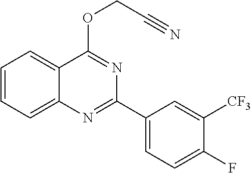 [SMILES: N#CCOc1nc(-c2ccc(F)c(C(F)(F)F)c2)nc2ccccc12] | GL-701 | <10⁻⁸ | | 0.25 | |
| 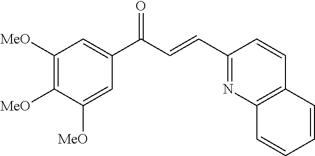 [SMILES: COc1cc(C(=O)/C=C/c2ccc3ccccc3n2)cc(OC)c1OC] | GL-713 | 0.55 | | 3 | |
| 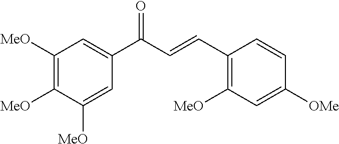 [SMILES: COc1ccc(/C=C/C(=O)c2cc(OC)c(OC)c(OC)c2)c(OC)c1] | GL-714 | 1.1 | | 5.2 | |
| 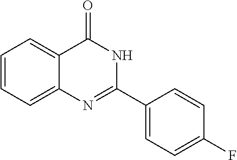 [SMILES: O=c1[nH]c(-c2ccc(F)cc2)nc2ccccc12] | GL-717 | 1.9 | | 3.9 | |
| 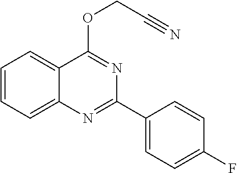 [SMILES: N#CCOc1nc(-c2ccc(F)cc2)nc2ccccc12] | GL-718 | 0.85 | | 0.015 | |
| 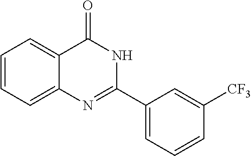 [SMILES: O=c1[nH]c(-c2cccc(C(F)(F)F)c2)nc2ccccc12] | GL-728 | 0.2 | | | |
| 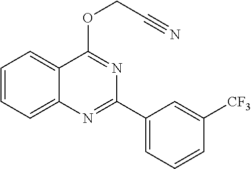 [SMILES: N#CCOc1nc(-c2cccc(C(F)(F)F)c2)nc2ccccc12] | GL-729 | 0.045 1 phase <0.00017 | | 0.05 | |

Table 4 includes additional potent CYP1B1 inhibitors that contain nitrile groups. These are coordinating group, and allow, for example, for bonding to a metal center. O-alkylation increases the potency of quinazolinone CYP inhibitors. This effect is enhanced by the addition of coordinating nitrile groups. The inhibitors maintain potency when tethering groups are included.

TABLE 4
CYP1B1 inhibitors that contain nitrile groups.

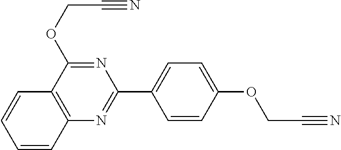 [SMILES: N#CCOc1ccc(-c2nc(OCC#N)c3ccccc3n2)cc1]

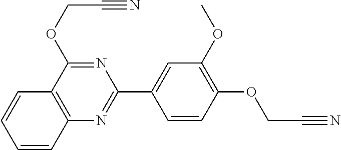 [SMILES: COc1cc(-c2nc(OCC#N)c3ccccc3n2)ccc1OCC#N]

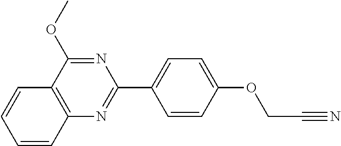 [SMILES: COc1nc(-c2ccc(OCC#N)cc2)nc2ccccc12]

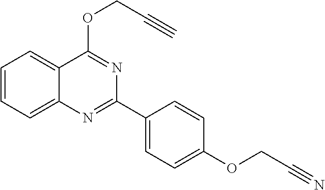 [SMILES: C#CCOc1nc(-c2ccc(OCC#N)cc2)nc2ccccc12]

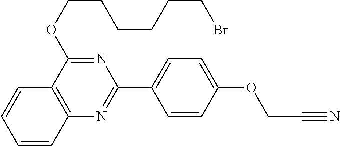 [SMILES: N#CCOc1ccc(-c2nc(OCCCCCCBr)c3ccccc3n2)cc1]

### Example 6

CYP Inhibitors were studied for triggered activation. Modification of CYP-selective inhibitors to contain heteroatoms allows for incorporation of metal complexes through coordinative bonds. In this context, the metal complexes themselves serve as protecting groups, in order to inactivate the inhibitor until a specific stimulus causes the breaking of the coordinative bond. The metal complex may also have biological activity once separated from the inhibitor.

Stilbene inhibitors were generated with coordinating groups to establish structure-activity relationships. Illustrative compounds are shown in Table 5. Compound 1 and 7 are non-coordinating control compounds that exhibit poor activity.

TABLE 5
Examples of Stilbene Inhibitors

| | |
|---|---|
| 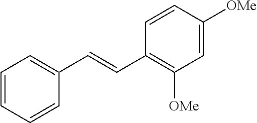 [SMILES: COc1ccc(/C=C/c2ccccc2)c(OC)c1] | 1 |
| 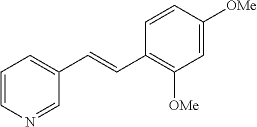 [SMILES: COc1ccc(/C=C/c2cccnc2)c(OC)c1] | 2 |
| 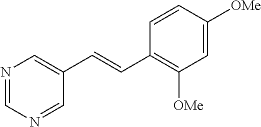 [SMILES: COc1ccc(/C=C/c2cncnc2)c(OC)c1] | 3 |
| 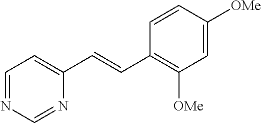 [SMILES: COc1ccc(/C=C/c2ccncn2)c(OC)c1] | 4 |
| 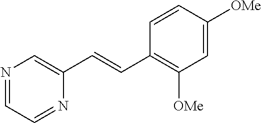 [SMILES: COc1ccc(/C=C/c2cnccn2)c(OC)c1] | 5 |
| 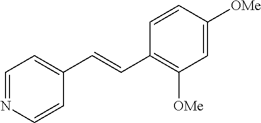 [SMILES: COc1ccc(/C=C/c2ccncc2)c(OC)c1] | 6 |
| 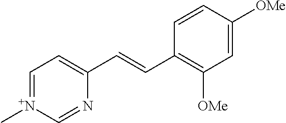 [SMILES: COc1ccc(/C=C/c2cc[n+](C)cn2)c(OC)c1] | 7 |
| 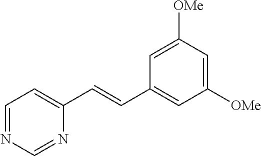 [SMILES: COc1cc(/C=C/c2ccncn2)cc(OC)c1] | 8 |
| 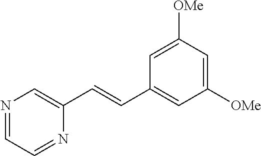 [SMILES: COc1cc(/C=C/c2cnccn2)cc(OC)c1] | 9 |
| 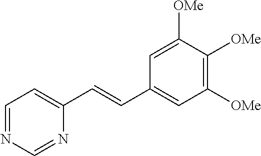 [SMILES: COc1cc(/C=C/c2ccncn2)cc(OC)c1OC] | 10 |
| 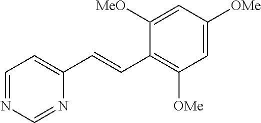 [SMILES: COc1cc(OC)c(/C=C/c2ccncn2)c(OC)c1] | 11 |
| 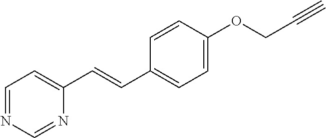 [SMILES: C#CCOc1ccc(/C=C/c2ccncn2)cc1] | 12 |
| 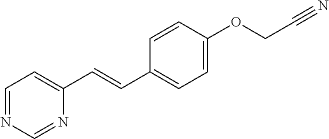 [SMILES: N#CCOc1ccc(/C=C/c2ccncn2)cc1] | 13 |
| 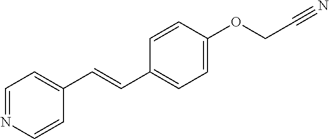 [SMILES: N#CCOc1ccc(/C=C/c2ccncc2)cc1] | 14 |

Coordination of potent systems, such as 4, 6, and 13, to metal centers produced systems where the CYP inhibitor was prevented from engaging with the CYP. This turns the CYP inhibitor into a prodrug. For example, the coordination of specific ruthenium complexes creates compounds that are less active under dark conditions, but can be activated with visible light, including wavelengths in the blue, green, red, and IR regions. Based on prior work, ruthenium (II) complexes were synthesized that incorporated stilbene CYP1B1 inhibitors containing coordinating heterocycles. These ruthenium complexes may contain one or more of the CYP inhibitors, and different co-ligands. These include, but are not limited to, 2,2′-bipyridine, 1,10-phenanthroline, 2,2′-biquinoline, 2 2′-biquinoline-4 4′-dicarboxylic acid, 2,2′;6′,2″-terpyridine, and derivatives thereof. Examples are provided below, where the stilbene inhibitor is shown in red in the first structure. Different overall charges on the complex can be produced, depending on the co-ligands. In addition, different counterions (such as Cl—, PF₆—, NO₃—, BPh₄-, BF₄—) can be used to adjust physiochemical properties such as solubility and cellular uptake.

TABLE 6
Example Ru(II) complexes containing CYP inhibitors.

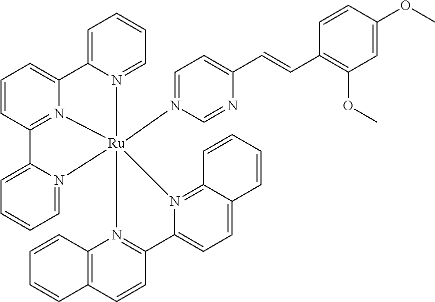

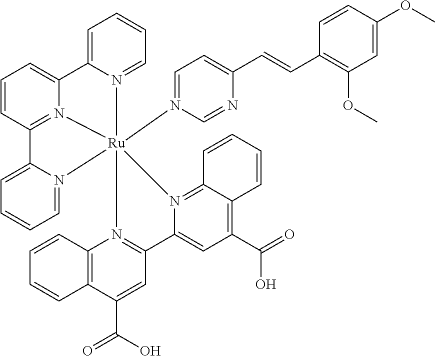

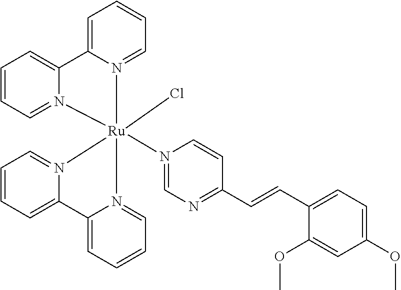

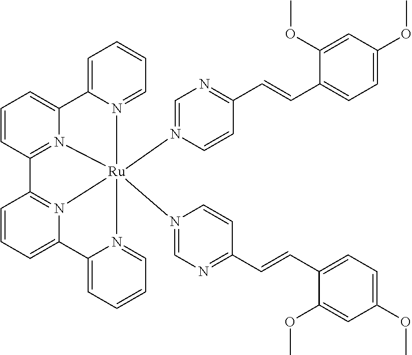

The exemplary structures set forth in Table 6 contain inhibitor GL433, listed a compound 4 in Table 5. More potent analogues have been generated, for example, with compound 6.

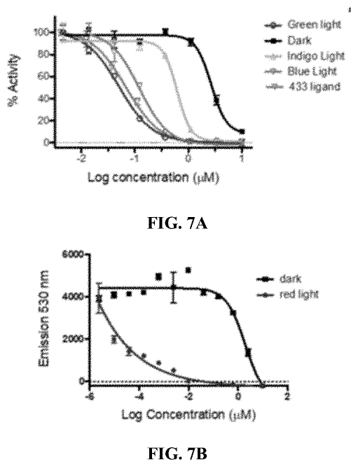

Various systems exhibited desired properties, including low CYP inhibition in the dark and enhanced activity following irradiation. Two examples are shown in FIGS. 7A and 7B. The first can be activated with various wavelengths of light due to an absorption in the blue to green region of the spectrum. The second was designed for red light activation to facilitate deeper penetration of photons into tissues.

Another application is activation by ionizing radiation, such as diagnostic or therapeutic x-rays and gamma radiation. In addition, the metal may be used to enhance cell specific targeting through conjugation of moieties to enhance cell-specific uptake. Notably, various coordinating groups may be used. A coordinating group is defined as a Lewis Base, and contains a lone pair of electrons. These electrons can be donated to the metal center, which is a Lewis Acid. Coordinating groups include, but are not limited to, all nitrogen-containing heterocycles, nitriles, thiols and thioethers, oxygen containing systems such as carbonyl and carboxylates, and carbenes. Alternative metal centers may be used for activation by reduction within cells due to redox homeostasis modifications. Examples include Co(III) or Pt(IV) complexes. Additional systems containing Os(II) and Cu(I/II) are being explored. In each case, the concept is to achieve activation, defined as ligand release, with photons, electrons, or ionizing radiation. This approach also works for other CYP inhibitors that contain coordinating groups.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Faiq, M. A.; Dada, R.; Sharma, R.; Saluja, D.; Dada, T. CYP1B1: a unique gene with unique characteristics. Curr Drug Metab 2014, 15 (9), 893-914.
2. Aklillu, E.; Oscarson, M.; Hidestrand, M.; Leidvik, B.; Otter, C.; Ingelman-Sundberg, M. Functional analysis of six different polymorphic CYP enzyme variants found in an Ethiopian population. Mol Pharmacol 2002, 61 (3), 586-94.
3. Vasiliou, V.; Gonzalez, F. J. Role of CYP1B1 in glaucoma. Annu Rev Pharmacol Toxicol 2008, 48, 333-58.
4. Choudhary, D.; Jansson, I.; Schenkman, J. B. CYP1B1, a developmental gene with a potential role in glaucoma therapy. Xenobiotica 2009, 39 (8), 606-15.
5. Wiggs, J. L.; Langgurth, A. M.; Allen, K. F. Carrier frequency of CYP1B1 mutations in the United States (an American Ophthalmological Society thesis). Trans Am Ophthalmol Soc 2014, 112, 94-102.
6. Horley, N. J.; Beresford, K. J.; Chawla, T.; McCann, G. J.; Ruparelia, K. C.; Gatchie, L.; Sonawane, V. R.; Williams, I. S.; Tan, H. L.; Joshi, P.; Bharate, S. S.; Kumar, V.; Bharate, S. B.; Chaudhuri, B. Discovery and characterization of novel CYP1B1 inhibitors based on heterocyclic chalcones: Overcoming cisplatin resistance in CYP1B1-overexpressing lines. Eur. J. Med. Chem. 2017, 129, 159-174.
7. Huang, R. S.; Duan, S.; Kistner, E. O.; Bleibel, W. K.; Delaney, S. M.; Fackenthal, D. L.; Das, S.; Dolan, M. E. Genetic variants contributing to daunorubicin-induced cytotoxicity. Cancer Res 2008, 68 (9), 3161-8.
8. Chang, I.; Mitsui, Y.; Fukuhara, S.; Gill, A.; Wong, D. K.; Yamamura, S.; Shahryari, V.; Tabatabai, Z. L.; Dahiya, R.; Shin, D. M.; Tanaka, Y. Loss of miR-200c up-regulates CYP1B1 and confers docetaxel resistance in renal cell carcinoma. Oncotarget 2015, 6 (10), 7774-87.
9. De Iullis, F.; Salerno, G.; Taglieri, L.; Scarpa, S. Are pharmacogenomic biomarkers an effective tool to predict taxane toxicity and outcome in breast cancer patients? Literature review. Cancer Chemother Pharmacol 2015, 76 (4), 679-90.
10. Dumont, A.; Pannier, D.; Ducoulombier, A.; Tresch, E.; Chen, J.; Kramar, A.; Revillion, F.; Peyrat, J. P.; Bonneterre, J. ERCC1 and CYP1B1 polymorphisms as predictors of response to neoadjuvant chemotherapy in estrogen positive breast tumors. Springerplus 2015, 4, 327.
11. Sissung, T. M.; Danesi, R.; Price, D. K.; Steinberg, S. M.; de Wit, R.; Zahid, M.; Gaikwad, N.; Cavalieri, E.; Dahut, W. L.; Sackett, D. L.; Figg, W. D.; Sparreboom, A. Association of the CYP1B1*3 allele with survival in patients with prostate cancer receiving docetaxel. Mol Cancer Ther 2008, 7 (1), 19-26.
12. Bansal, S.; Leu, A. N.; Gonzalez, F. J.; Guengerich, F. P.; Chowdhury, A. R.; Anandatheerthavarada, H. K.; Avadhani, N. G. Mitochondrial targeting of cytochrome P450 (CYP) 1B1 and its role in polycyclic aromatic hydrocarbon-induced mitochondrial dysfunction. J Biol Chem 2014, 289 (14), 9936-51.
13. Verma, S.; Saxena, R.; Siddiqui, M. H.; Santha, K.; Sethupathy, S. Evaluation of CYP1B1 Expression, Oxidative Stress and Phase 2 Detoxification Enzyme Status in Oral Squamous Cell Carcinoma Patients. J Clin Diagn Res 2017, 11 (3), BC01-BC05.
14. Maayah, Z. H.; Althurwi, H. N.; Abdelhamid, G.; Lesyk, G.; Jurasz, P.; El-Kadi, A. O. CYP inhibition attenuates doxorubicin-induced cardiotoxicity through a mid-chain HETEs-dependent mechanism. Pharmacol. Res. 2016, 105, 28-43.
15. Gajjar, K.; Martin-Hirsch, P. L.; Martin, F. L. CYP and hormone-induced cancer. Cancer Lett 2012, 324 (1), 13-30.
16. Hayes, C. L.; Spink, D. C.; Spink, B. C.; Cao, J. Q.; Walker, N. J.; Sutter, T. R. 17 beta-estradiol hydroxylation catalyzed by human cytochrome P450 1B1. Proc Natl Acad Sci USA 1996, 93 (18), 9776-81.
17. Tsuchiya, Y.; Nakajima, M.; Yokoi, T. Cytochrome P450-mediated metabolism of estrogens and its regulation in human. Cancer Lett 2005, 227 (2), 115-24.
18. Nishida, C. R.; Everett, S.; Ortiz de Montellano, P. R. Specificity determinants of CYP1B1 estradiol hydroxylation. Mol Pharmacol 2013, 84 (3), 451-8.
19. Reis, L. M.; Tyler, R. C.; Weh, E.; Hendee, K. E.; Kariminejad, A.; Abdul-Rahman, O.; Ben-Omran, T.; Manning, M. A.; Yesilyurt, A.; McCarty, C. A.; Kitchner, T. E.; Costakos, D.; Semina, E. V. Analysis of CYP1B1 in pediatric and adult glaucoma and other ocular phenotypes. Mol Vis 2016, 22, 1229-1238.
20. Zhao, Y.; Wang, S.; Sorenson, C. M.; Teixeira, L.; Dubielzig, R. R.; Peters, D. M.; Conway, S. J.; Jefcoate, C. R.; Sheibani, N. Cyp1b1 mediates periostin regulation of trabecular meshwork development by suppression of oxidative stress. Mol Cell Biol 2013, 33 (21), 4225-40.
21. Li, F.; Zhu, W.; Gonzalez, F. J. Potential role of CYP1B1 in the development and treatment of metabolic diseases. Pharmacol Ther 2017, 178, 18-30.
22. Hodgson, J. ADMET—turning chemicals into drugs. Nature Biotechnology 2001, 19, 722-726.
23. Mu, W.; Hu, C.; Zhang, H.; Qu, Z.; Cen, J.; Qiu, Z.; Li, C.; Ren, H.; Li, Y.; He, X.; Shi, X.; Hui, L. miR-27b synergizes with anticancer drugs via p53 activation and CYP1B1 suppression. Cell Res 2015, 25 (4), 477-95.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound of the formula: 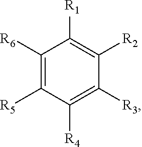
wherein R1, R4, R5, and R6 are independently selected from the group consisting of H, OH, alkoxy, thiazole, phenylthiazole, halo, alkylhalo, alkyl, alkenyl, alkynyl, alkenylpyrimidinyl, alkenylpyrazinyl, alkenylpyridinyl, cyano, alkyoxycyano, amide, benzimidazole, and alkylketone; and
R2 and R3 are independently selected from the group consisting of H, OH, alkoxy, thiazole, phenylthiazole, halo, alkylhalo, alkyl, alkenyl, alkynyl, alkenylpyrimidinyl, alkenylpyrazinyl, alkenylpyridinyl, cyano, alkyoxycyano, amide, benzimidazole, and alkylketone; or R2 and R3 taken together with the atoms to which they are bound for a 6 member substituted or unsubstituted heterocycle;
wherein at least one of R1-R6 is selected from the group consisting of substituted or unsubstituted thiazole, quinazolinone, (E)-4-(prop-1-en-1-yl)pyrimidine, (E)-2-(prop-1-en-1-yl)pyrazine, (E)-3-(prop-1-en-1-yl)pyridine, (E)-5-(prop-1-en-1-yl)pyrimidine, (E)-prop-1-en-1-ylbenzene, 1H-benzo[d]imidazole, quinazoline-4(3H)-one, 2-(methyleneamino)benzamide, and 4-alkoxy-3,4-dihydroquinazoline.

2. The compound of claim 1, selected from the compounds set forth in Table 2.

3. The compound of claim 1, selected from the compounds set forth in Table 3.

4. The compound of claim 1, selected from the compounds set forth in Table 4.

5. The compound of claim 1, wherein at least one of R1-R6 is a substituted moiety, wherein the substitution is a metal complex.

6. The compound of claim 3, selected from the compounds set forth in Table 5.

7. A method for inhibiting CYP by administering to cells the compound of claim 1.

8. A method for inhibiting CYP in a subject comprising administering to the subject a therapeutically effective amount of the compound of claim 1.

9. A method of restoring the chemotherapeutic efficacy of a chemotherapeutic agent comprising administering to a patient the compound of claim 1 together with the chemotherapeutic agent.
